(19) United States
(12) Patent Application Publication
(10) Pub. No.: US 2026/0199878 A1
(43) Pub. Date: Jul. 16, 2026

(54) CATALYST FOR SIMULTANEOUS CONVERSION REACTION OF SUGAR TO ALCOHOL AND ACID AND METHOD FOR SIMULTANEOUSLY PRODUCING ALCOHOL AND ACID FROM SUGAR BY USING SAME

(71) Applicant: KOREA RESEARCH INSTITUTE OF CHEMICAL TECHNOLOGY, Daejeon (KR)

(72) Inventors: Dong Won HWANG, Daejeon (KR); Seung Hyeok CHA, Daejeon (KR); Young Kyu HWANG, Daejeon (KR); Gwang Nam YUN, Daejeon (KR); Ji Hoon KIM, Daejeon (KR); Ma Eum LEE, Daejeon (KR); Ali AWAD, Daejeon (KR); In Yong EOM, Daejeon (KR)

(73) Assignee: KOREA RESEARCH INSTITUTE OF CHEMICAL TECHNOLOGY, Daejeon (KR)

(21) Appl. No.: 19/564,765

(22) Filed: Mar. 12, 2026

Related U.S. Application Data
(63) Continuation of application No. PCT/KR2024/017283, filed on Nov. 5, 2024.

(30) Foreign Application Priority Data
Nov. 8, 2023 (KR) 10-2023-0153825

(51) Int. Cl. B01J 23/63; B01J 21/04; B01J 21/06; B01J 23/42; B01J 23/60; B01J 35/30; B01J 35/61; B01J 35/63; B01J 35/64; B01J 37/03; B01J 37/04; B01J 37/08; B01J 37/18; C07C 29/149; C07C 51/09

(52) U.S. Cl. CPC B01J 23/63; B01J 21/04; B01J 21/063; B01J 21/066; B01J 23/42; B01J 23/60; B01J 35/394; B01J 35/613; B01J 35/633; B01J 35/647; B01J 37/035; B01J 37/04; B01J 37/088; B01J 37/18; C07C 29/149; C07C 51/09

(57) ABSTRACT

The present invention relates to a catalyst for simultaneously producing an alcohol and an acid from a sugar and a method for simultaneously producing an alcohol and an acid from a sugar by using same and, more specifically, to a catalyst that is easily recoverable and enables the stable and simultaneous production of an acid and an alcohol from a sugar under mild reaction conditions with low temperature and low pressure compared with existing reaction conditions, and to a method for simultaneously producing an alcohol and an acid from a sugar by using same.

### CROSS REFERENCE TO RELATED APPLICATIONS

The present application is a continuation of PCT International Application No. PCT/KR2024/017283, which has an International filing date of Nov. 5, 2024, and which claims priority to Korean Patent Application No. 10-2023-0153825, filed on Nov. 8, 2023, the entire contents of each of which is incorporated herein for all purposes by this reference.

### TECHNICAL FIELD

The present disclosure relates to a catalyst for a simultaneous conversion reaction of a sugar to an alcohol and an acid, and a method of simultaneously producing an alcohol and an acid from a sugar using the same. More specifically, the present disclosure relates to a catalyst used for a simultaneous conversion reaction of an alcohol and an acid from a sugar, and a method of simultaneously producing an alcohol and an acid from a sugar using the same.

### BACKGROUND ART

Fossil fuels such as petroleum, gas, and coal are finite resources, experiencing continuous price increases, and competition among nations to stably secure these resources is intensifying. Furthermore, chemical products produced from such fossil fuels generate greenhouse gases and waste as by-products during their production processes, leading to environmental pollution, which has become a factor contributing to the rapid contraction of the conventional chemical industry.

In Korea, which relies on imports for most of its energy, it is essential to establish long-term energy supply policies that can minimize reliance on energy imports, as well as to develop fundamentally clean alternative energy sources, in order to ensure national security and sustained economic growth. Under these circumstances, biomass has attracted attention as a field of alternative energy capable of addressing concerns over fossil fuel depletion and environmental pollution. Instability in petroleum supply resulting from petroleum resource depletion and prolonged high oil prices, along with strengthened environmental regulations such as restrictions on greenhouse gas emissions from fossil fuel usage, are acting as impediments to global economic growth. In particular, the chemical industry, which is most directly affected by high oil prices and environmental regulations, recognizes the reduction of reliance on fossil fuels and the transition to an environmentally friendly industrial structure as an important alternative.

Accordingly, there is a growing demand for the development of new environmentally friendly biochemical processes utilizing biomass as a feedstock, capable of replacing fossil fuel-based chemical processes, that is, processes that can minimize the consumption of fossil fuels and the production of waste harmful to humankind.

One of the notable recent movements in approaches for the development of environmentally friendly biochemical processes is the growing prominence of the biorefinery field utilizing biomass. The term biorefinery refers to a new concept in which biofuels and chemical products are produced exclusively from biomass through biological and chemical conversion processes without the use of fossil fuels. Despite historically originating from the term oil refinery, which refers to the refining of crude oil in the conventional petrochemical industry, the term biorefinery in fact signifies core technologies covering the entire lifecycle for producing all biochemical products, including biofuels, from biomass feedstocks through biological and chemical conversion processes.

Similarly, in oil refineries developed as comprehensive and integrated processes producing industrial/transportation fuels and all types of chemical products from crude oil, biorefineries are being developed and constructed as integrated processes based on technologies for producing biofuels such as ethanol, butanol, and acetone, as well as chemical feedstocks such as sorbitol, xylitol, lactic acid, and succinic acid, from biomass, and comprehensive plant systems to implement such technologies. In particular, interest in alcohols, such as sorbitol and xylitol, and acids, such as xylonic acid and gluconic acid, has increased significantly, as these compounds are widely used in food additives, pharmaceuticals, cosmetics, and the like.

Hydrogenation reactions of corresponding sugars mostly produce such alcohols, and representative examples are as follows. As hydrogenation methods, U.S. Pat. Nos. 3,586,537, 4,008,285, 6,414,201, and the like disclose methods of producing xylitol by subjecting xylose to hydrogenation using a Raney nickel catalyst. These hydrogenation methods are performed under high-temperature and high-pressure conditions, require catalyst recovery processes, and necessitate separate reactions and different catalysts to produce acids such as xylonic acid and gluconic acid in addition to sugar alcohols. This results in high production costs and is thus disadvantageous.

### DOCUMENTS OF RELATED ART

### Patent Documents

(Patent Document 1) U.S. Pat. No. 3,586,537 (registered on Jun. 22, 1971)
(Patent Document 2) U.S. Pat. No. 4,008,285 (registered on Feb. 15, 1977)
(Patent Document 3) U.S. Pat. No. 6,414,201 (registered on Jul. 2, 2002)

### DISCLOSURE

### Technical Problem

The present disclosure, which has been devised to solve the problems described above, primarily aims to provide a catalyst for the simultaneous conversion reaction of a sugar into an alcohol and an acid, the catalyst being readily recoverable and enabling the stable and simultaneous production of the acid and the alcohol from the sugar under mild reaction conditions at reduced temperature and pressure compared to conventional reaction conditions.

In addition, another objective of the present disclosure is to provide a method of simultaneously producing an alcohol and an acid from a sugar in the presence of the above catalyst and a base.

### Technical Solution

In order to solve the problems described above, the present disclosure provides a catalyst for a simultaneous conversion reaction of a sugar to an acid and an alcohol to simultaneously produce the acid and the alcohol from the sugar in the presence of a base.

The catalyst may be characterized by having a form in which platinum serving as a catalytically active metal is supported on a metal oxide containing one or more metals selected from the group consisting of zirconium (Zr), aluminum (Al), cerium (Ce), zinc (Zn), titanium (Ti), hafnium (Hf), nickel (Ni), cobalt (Co), iron (Fe), manganese (Mn), aluminum (Al), gallium (Ga), and indium (In), the metal oxide serving as a catalyst support.

The base may be a hydroxide of an alkali metal and/or a hydroxide of an alkaline earth metal, and may be present in an amount of 1 mol or more with respect to 1 mol of the sugar.

The platinum may be present in an amount in a range of 0.1 to 20 wt % with respect to the total weight of the catalyst, and the sugar in the reaction may have a concentration of 1 mol or more.

The sugar may be one or more selected from the group consisting of erythrose, ribose, arabinose, xylose, lyxose, allose, altrose, glucose, mannose, galactose, talose, hexose, maltose, lactose, fructose, lactulose, isomaltulose, rhamnose, sucrose, starch sugars, starch hydrolysates, cellulose hydrolysates, and hemicellulose hydrolysates.

In addition, the present disclosure provides a method of simultaneously producing an acid and an alcohol from a sugar by subjecting the sugar to a reaction in the presence of a base and a catalyst, wherein the catalyst is characterized by having a form in which platinum serving as a catalytically active metal is supported on a metal oxide containing one or more metals selected from the group consisting of zirconium (Zr), aluminum (Al), cerium (Ce), zinc (Zn), titanium (Ti), hafnium (Hf), nickel (Ni), cobalt (Co), iron (Fe), manganese (Mn), aluminum (Al), gallium (Ga), and indium (In), the metal oxide serving as a catalyst support.

In the production method of the present disclosure, the base may be a hydroxide of an alkali metal and/or a hydroxide of an alkaline earth metal, the base may be used in an amount of 1 mol or more with respect to 1 mol of the sugar, the sugar in the reaction may have a concentration of 1 mol or more, the platinum may be present in an amount in a range of 0.1 to 20 wt % with respect to the total weight of the catalyst, and the reaction may be performed at a temperature in a range of 0° C. to 200° C.

### Advantageous Effects

A catalyst according to the present disclosure uses Pt as an active metal and a metal oxide as a support, thereby enabling the simultaneous production of an acid and an alcohol from a sugar under mild single-reaction conditions at reduced temperature and pressure. At the same time, compared to conventional Pt/C catalysts, the catalyst is advantageously recoverable and reusable even under strongly basic conditions.

Thus, the catalyst of the present disclosure can exhibit stable recycling efficiency over an extended period during use.

Furthermore, whereas alcohols and acids have been conventionally produced separately from sugars in the presence of different catalysts, the present disclosure enables the simultaneous production of an alcohol and an acid under mild single-reaction conditions at reduced temperature and pressure. As a result, conventional alcohol and acid production processes that are complex and time-consuming can be shortened, thereby improving the productivity of alcohol and acid production from sugars.

### DESCRIPTION OF DRAWINGS

FIG. 1 illustrates a conversion reaction mechanism of glucose according to one embodiment of the present disclosure.

FIG. 2 illustrates a conversion reaction mechanism of xylose according to one embodiment of the present disclosure.

FIG. 3 shows an X-ray diffraction (XRD) pattern measurement graph of catalysts prepared in Preparation Examples 1 and 3 to 5 of the present disclosure.

FIG. 4 shows graphs illustrating results of catalytic activity measurements upon reuse of catalysts prepared in Preparation Examples 4 and 10 of the present disclosure, wherein (a) shows a graph illustrating measurements of xylose conversion rate and yields of xylonic acid and xylitol for the catalyst of Preparation Example 4, and (b) shows a graph illustrating measurements of xylose conversion rate and yields of xylonic acid and xylitol for the catalyst of Preparation Example 10.

FIG. 5 shows scanning electron microscope (SEM) images of catalysts prepared in Preparation Examples 4 and 10 of the present disclosure, the images taken before and after a reaction, wherein (a) shows an SEM image of the catalyst of Preparation Example 4 before the reaction, (b) shows an SEM image of the catalyst of Preparation Example 4 after the reaction, (c) shows an SEM image of the catalyst of Preparation Example 10 before the reaction, and (b) shows an SEM image of the catalyst of Preparation Example 10 after the reaction.

### MODE FOR INVENTION

Unless defined otherwise, all technical and scientific terms used herein have the same meaning as commonly understood by those skilled in the art to which the present disclosure pertains. Generally, the nomenclature used herein is well-known and commonly used in the art.

As stated herein, the terms such as “comprising”, “including”, or “having” are intended to specify the presence of features, integers, steps, operations, components, parts, or combinations thereof stated herein but do not exclude the possibility that other unmentioned features, integers, steps, operations, components, parts, or combinations thereof may be present or added.

Unless otherwise specified, the reaction and mixing to be described below may be carried out under conventional reaction and mixing conditions, typically at room temperature and atmospheric pressure, without additional modifications.

However, the interpretation of the present disclosure should not extend beyond what is clearly understood by those skilled in the art.

The present disclosure relates to a catalyst used in a reaction for simultaneously producing an acid and an alcohol by subjecting a sugar to a conversion reaction in the presence of a base, and a method of simultaneously producing an acid and an alcohol from a sugar using the same. In this case, the sugar conversion reaction involves reacting a sugar in the presence of a base and a catalyst, thereby simultaneously producing an acid and an alcohol. Additionally, the catalyst is characterized by having a form in which platinum is supported as a catalytically active metal on a specific metal oxide catalyst support.

The catalytically active metal platinum (Pt) may be supported in an amount in the range of 0.1 to 20 wt % with respect to the total weight of the catalyst, which is supported in an amount preferably in the range of 0.1 to 10 wt % and more preferably in the range of 1 to 8 wt %. When the amount of platinum is less than 0.1 wt % with respect to the total weight of the catalyst, active components may be insufficiently present in the sugar conversion reaction, resulting in reduced reactivity, which is problematic. When the amount of platinum exceeds 20 wt %, the dispersion of platinum decreases, and thus the effect of increasing the activity relative to the amount of platinum is comparatively low. Additionally, C—C bond cleavage may rather occur, leading to reduced selectivity. Accordingly, the effect of increasing the activity relative to the platinum content is insignificant, which may be economically disadvantageous.

In addition, the catalyst support may be a metal compound containing one or more metals selected from the group consisting of titanium (Ti), zirconium (Zr), cerium (Ce), hafnium (Hf), zinc (Zn), nickel (Ni), cobalt (Co), iron (Fe), manganese (Mn), aluminum (Al), gallium (Ga), and indium (In) in terms of excellent stability even under basic conditions, the conversion rate of the sugar, and the selectivity toward the acid and alcohol. From the viewpoint of high long-term stability, the catalyst support preferably contains zirconium (Zr), cerium (Ce), titanium (Ti), hafnium (Hf), and the like.

In this case, the metal compound of the catalyst support may be a metal oxide represented by MO (where M═Zn, etc.), MO₂ (where M═Ti, Zr, Ce, Hf, etc.), M₂O₃ (where M═Al, etc.), MOOH (where M═Ni, Co, Fe, Mn, Al, Ga, In, etc.), and the like.

In the meantime, the catalyst according to the present disclosure is applicable without limitation, provided that a method capable of supporting platinum on the catalyst support is used. Specifically, platinum may be supported on the catalyst support using methods known in the art to which the present disclosure pertains, including an impregnation method, a co-precipitation method, a solid-phase supporting method, a vapor deposition method, a wash-coating method, a sol-gel method, and a hydrothermal synthesis method.

In one embodiment, supporting by impregnation includes a step of impregnating the catalyst support with a platinum precursor solution and a step of drying and calcining the catalyst support on which the platinum precursor solution is supported.

The impregnation may be performed by dissolving a platinum precursor in a solvent and then uniformly dispersing the platinum precursor dissolved in the solvent onto the catalyst support in multiple steps, followed by wet impregnation performed at a temperature in the range of 10° C. to 200° C. The supporting time may vary depending on conditions, provided that sufficient support is achieved, and may, for example, range from 1 to 12 hours.

The platinum precursor may be one or more selected from platinum-containing nitrates, sulfates, phosphoric acid, halides, alkoxides, oxynitrates, hydroxides, acetates, alkyl salts, hydrates thereof, and the like. Preferably, a hydrated halide may be used. As for the solvent, any known solvent capable of dissolving the platinum precursor, including water, glycol-based solvents, such as ethylene glycol, 1,2-propylene glycol, 1,3-butanediol, 1,4-butanediol, neopentyl glycol, diethylene glycol, 3-methyl-1,5-pentanediol, 1,6-hexanediol, and trimethylolpropane, or alcohol-based solvents, such as methanol, ethanol, isopropyl alcohol (IPA), and butanol, can be used for preparation. The solvent may be used in any content that can uniformly disperse the platinum precursor, without limitation. For example, the solvent content may be in the range of 100 to 500 parts by weight with respect to 100 parts by weight of the platinum precursor.

Subsequently, the catalyst support on which platinum is supported may be dried and calcined by adjusting time and temperature depending on conditions such as the supported amount of the catalytically active metal, the size of the catalyst support, and the amount thereof. For example, the catalyst support may be dried at a temperature in the range of 10° C. to 100° C. and then calcined at a temperature in the range of 400° C. to 1,100° C. for 3 to 10 hours. Additionally, during the calcination process, it is preferable to gradually increase the temperature for preparation, since abrupt temperature changes may cause rapid solvent evaporation and oxidation, leading to the formation of cracks and pores and a significant reduction in strength.

Thereafter, the calcined catalyst may be subjected to an activation step before the sugar conversion reaction. Specifically, the activation step may involve reduction at a temperature at which the catalyst is reducible (in the range of 100° C. to 600° C.) under a reducing atmosphere to activate the catalyst. In one embodiment, after loading the catalyst into a reactor, the catalyst may be reduced by introducing 1 to 10% hydrogen (H₂) gas at a flow rate in the range of 50 to 300 mL/min for 0.5 to 4 hours at a temperature in the range of 100° C. to 600° C.

After loaded into the reactor, the reduced catalyst may be subjected to the sugar conversion reaction in the presence of a base. The sugar conversion reaction according to the present disclosure may be performed in an open reactor or a closed reactor, and may be performed in a batch mode, a continuous mode, or the like.

The sugar used as a feedstock in this case may include monosaccharides, disaccharides, and polysaccharides. Specifically, the sugar may include a sugar selected from the group consisting of erythrose, arabinose, xylose, glucose, mannose, galactose, talose, maltose, lactose, fructose, lactulose, isomaltulose, rhamnose, sucrose, ribose, lyxose, allose, altrose, hexose, starch sugar, starch hydrolysates, cellulose hydrolysates, and hemicellulose hydrolysates, or mixtures of one or more sugars selected from the foregoing. From the viewpoint of reactivity, the sugar is preferably xylose, glucose, galactose, mannose, lactose, arabinose, or the like.

Since sugars are generally in a solid state at room temperature, it is preferable to dissolve the sugar in a suitable solvent for use to improve reaction efficiency. Although the solvent used for this purpose is not particularly limited to one that can dissolve the sugar serving as a feedstock, it is generally preferable to use water or alcohol alone, or a mixture thereof. Examples of the alcohol may include methanol, ethanol, propanol, isopropanol, or mixtures thereof. More preferably, water alone or a mixture of water and ethanol is used. When such a solvent is used, the concentration of the sugar in the solution is not particularly limited, but may be in the range of 1 to 60 wt % with respect to the total weight of the solvent and the sugar.

In addition, the concentration of the sugar may be 0.1 mol or more, and is preferably in the range of 1 to 3 mol. When the concentration of the sugar is less than 0.1 mol, the amount of hydrogen produced during the dehydrogenation reaction of the sugar is small, which may lead to a problem in that the alcohol yield is low.

To improve reaction efficiency, the sugar conversion reaction may be performed under strongly alkaline conditions at a pH of 13 or higher by adding a base. The base added in this case may be a hydroxide of an alkali metal or a hydroxide of an alkaline earth metal. Specifically, the base may be one or more selected from KOH, NaOH, LiOH, CsOH, RbOH, Ca(OH)₂, Mg(OH)₂, Sr(OH)₂, Ba(OH)₂, NH₄OH, and the like, and is preferably KOH, NaOH, or the like.

In this case, the base may be used in an amount of 0.5 mol or more with respect to 1 mol of the sugar. Preferably, the base is used in an amount in the range of 0.5 to 3 mol from the viewpoint of reaction activity. When using the base in an amount of less than 0.5 mol with respect to 1 mol of the sugar, the dehydrogenation efficiency of the sugar is reduced, which may lead to a problem in that the acid and alcohol yields are reduced.

The sugar conversion reaction using the catalyst of the present disclosure enables the simultaneous production of an acid and an alcohol from a sugar under mild single conditions compared to conventional catalysts.

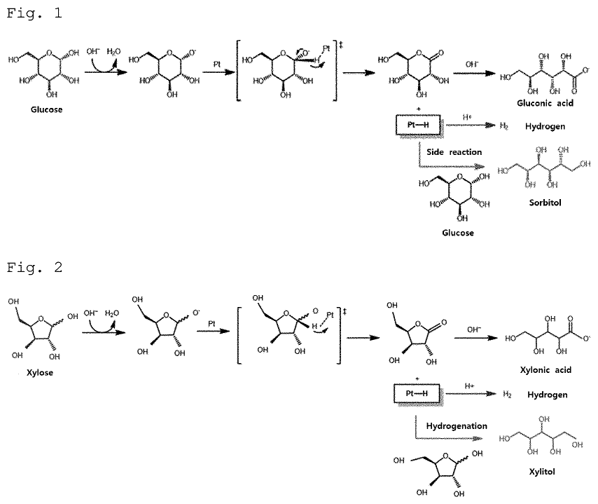

For example, in the sugar conversion reaction, as illustrated in FIGS. 1 and 2, gluconic acid and sorbitol may be simultaneously produced through the conversion reaction of glucose in the presence of a base and a catalyst when the sugar is glucose. Additionally, when the sugar is xylose, xylonic acid and xylitol may be simultaneously produced through the conversion reaction of xylose in the presence of a base and a catalyst.

The sugar conversion reaction herein may be performed at a temperature in the range of 0° C. to 200° C., and is more preferably performed at a temperature in the range of 0° C. to 60° C. When the reaction temperature is lower than 0° C., energy consumption associated with such a low-temperature process increases. When the reaction temperature exceeds 200° C., side reactions such as C—C cleavage reactions may increase.

As described above, the acid and alcohol produced according to the method of the present disclosure may be simultaneously produced from the sugar under mild single conditions at reduced temperature and pressure compared to conventional methods, without dissolution of catalyst components. As a result, by-products and waste are hardly generated, and the products may be obtained without complex separation processes. Furthermore, the catalyst participating in the reaction is recoverable and repeatedly usable in the sugar conversion reaction. Even upon repeated reuse, the catalyst can be used stably without dissolving or deactivating the catalyst components.

Hereinafter, the catalytic activity of the catalyst for the simultaneous conversion reaction of a sugar to an alcohol and an acid according to the present disclosure will be described through the following examples. For reference, although the following examples are provided to illustrate one or more preferred embodiments of the present disclosure, the present disclosure is not limited thereto. Various modifications may be made to the following examples within the scope of the present disclosure.

### Preparation Example: Preparation of Catalyst

### Preparation Example 1: Preparation of 1 wt % Pt/ZrO2 Catalyst

Zr(OH)₂ powder was calcined at 600° C. to obtain monoclinic ZrO₂, followed by dispersing 4.95 g of the obtained ZrO₂ in 50 mL of deionized water to prepare a ZrO₂ slurry. Subsequently, 0.13 g of H₂PtCl₆·6H₂O was dissolved in 30 mL of deionized water to obtain a platinum precursor solution. The obtained platinum precursor solution was slowly added to the ZrO₂ slurry under constant stirring to obtain a mixture. The obtained mixture was dried in an oven at 100° C. for 24 hours and then calcined in a calcination furnace at 500° C. for 4 hours to prepare a Pt/ZrO₂ catalyst on which 1 wt % of Pt was supported. Thereafter, the prepared catalyst was reduced at 300° C. for 2 hours under a 5% H₂/Ar flow to activate the catalyst.

### Preparation Example 2: Preparation of 2 wt % Pt/ZrO2 Catalyst

A Pt/ZrO₂ catalyst was prepared in the same manner as in Preparation Example 1, except that a platinum precursor solution in which 0.26 g of H₂PtCl₆·6H₂O was dissolved in 30 mL of deionized water was slowly added to the ZrO₂ slurry, thereby preparing the Pt/ZrO₂ catalyst on which 2 wt % of Pt was supported.

### Preparation Example 3: Preparation of 3 wt % Pt/ZrO2 Catalyst

A Pt/ZrO₂ catalyst was prepared in the same manner as in Preparation Example 1, except that a platinum precursor solution in which 0.39 g of H₂PtCl₆·6H₂O was dissolved in 30 mL of deionized water was slowly added to the ZrO₂ slurry, thereby preparing the Pt/ZrO₂ catalyst on which 3 wt % of Pt was supported.

### Preparation Example 4: Preparation of 5 wt % Pt/ZrO2 Catalyst

A Pt/ZrO₂ catalyst was prepared in the same manner as in Preparation Example 1, except that a platinum precursor solution in which 0.65 g of H₂PtCl₆·6H₂O was dissolved in 30 mL of deionized water was slowly added to the ZrO₂ slurry, thereby preparing the Pt/ZrO₂ catalyst on which 5 wt % of Pt was supported.

### Preparation Example 5: Preparation of 9 wt % Pt/ZrO2 Catalyst

A Pt/ZrO₂ catalyst was prepared in the same manner as in Preparation Example 1, except that a platinum precursor solution in which 1.17 g of H₂PtCl₆·6H₂O was dissolved in 60 mL of deionized water was slowly added to the ZrO₂ slurry, thereby preparing the Pt/ZrO₂ catalyst on which 9 wt % of Pt was supported.

### Preparation Example 6: Preparation of 5 wt % Pt/CeO2 Catalyst

Ce(CH₃COO)₃·xH₂O powder was calcined at 600° C. to obtain CeO₂, followed by dispersing 4.75 g of the obtained CeO₂ in 50 mL of deionized water to prepare a CeO₂ slurry. Subsequently, 0.65 g of H₂PtCl₆·6H₂O was dissolved in 30 mL of deionized water to obtain a platinum precursor solution. The obtained platinum precursor solution was slowly added to the CeO2 slurry under constant stirring to obtain a mixture. The obtained mixture was stirred at room temperature for 6 hours, followed by evaporation of water to recover a solid. The recovered solid was dried in an oven at 100° C. for 24 hours and then calcined in a calcination furnace at 500° C. for 4 hours to prepare a Pt/CeO₂ catalyst on which 5 wt % of Pt was supported. Thereafter, the prepared catalyst was reduced at 300° C. for 2 hours under a 5% H₂/Ar flow to activate the catalyst.

### Preparation Example 7: Preparation of 5 wt % Pt/Al2O3 Catalyst

A γ-Al₂O₃ slurry was prepared by dispersing 4.75 g of gamma-alumina (γ-Al₂O₃) in 90 mL of deionized water. Subsequently, 0.65 g of H₂PtCl₆·6H₂O was dissolved in 30 mL of deionized water to obtain a platinum precursor solution. The obtained platinum precursor solution was slowly added to the γ-Al₂O₃ slurry under constant stirring to obtain a mixture. The obtained mixture was stirred at room temperature for 6 hours, followed by evaporation of water to recover a solid. The recovered solid was dried in an oven at 100° C. for 24 hours and then calcined in a calcination furnace at 500° C. for 4 hours to prepare a Pt/Al₂O₃ catalyst on which 5 wt % of Pt was supported. Thereafter, the calcined catalyst was reduced at 300° C. for 2 hours under a 5% H₂/Ar flow to activate the catalyst.

### Preparation Example 8: Preparation of 5 wt % Pt/ZnO Catalyst

A ZnO₂ slurry was prepared by dispersing 4.75 g of ZnO₂ in 40 mL of deionized water. Subsequently, 0.65 g of H₂PtCl₆·6H₂O was dissolved in 30 mL of deionized water to obtain a platinum precursor solution. The obtained platinum precursor solution was slowly added to the ZnO₂ slurry under constant stirring to obtain a mixture. The obtained mixture was stirred at room temperature for 6 hours, followed by evaporation of water to recover a solid. The recovered solid was dried in an oven at 100° C. for 24 hours and then calcined in a calcination furnace at 500° C. for 4 hours to prepare a Pt/ZnO catalyst on which 5 wt % of Pt was supported. Thereafter, the calcined catalyst was reduced at 300° C. for 2 hours under a 5% H₂/Ar flow to activate the catalyst.

### Preparation Example 9: Preparation of 5 wt % Pt/TiO2 Catalyst

A TiO₂ slurry was prepared by dispersing 4.75 g of TiO₂ in 40 mL of deionized water. Subsequently, 0.65 g of H₂PtCl₆·6H₂O was dissolved in 30 mL of deionized water to obtain a platinum precursor solution. The obtained platinum precursor solution was slowly added to the TiO₂ slurry under constant stirring to obtain a mixture. The obtained mixture was stirred at room temperature for 6 hours, followed by evaporation of water to recover a solid. The recovered solid was dried in an oven at 100° C. for 24 hours and then calcined in a calcination furnace at 500° C. for 4 hours to prepare a Pt/TiO₂ catalyst on which 5 wt % of Pt was supported. Thereafter, the calcined catalyst was reduced at 300° C. for 2 hours under a 5% H₂/Ar flow to activate the catalyst.

### Preparation Example 10: Preparation of 5 wt % Pt/C Catalyst

A Pt/C catalyst (CAS No. 7440-06-4) on which 5 wt % of Pt was supported, purchased from Sigma-Aldrich, was used.

### Preparation Example 11: Preparation of 5 wt % Ru/ZrO2 Catalyst

Zr(OH)₄ powder was calcined at 600° C. to obtain ZrO₂, followed by dispersing 4.75 g of the obtained ZrO₂ in 40 mL of deionized water to prepare a ZrO₂ slurry. Subsequently, 0.51 g was dissolved in 30 mL of deionized water to obtain a ruthenium precursor solution. The obtained ruthenium precursor solution was slowly added to the ZrO₂ slurry under constant stirring to obtain a mixture. The obtained mixture was stirred at room temperature for 6 hours, followed by evaporation of water to recover a solid. The recovered solid was dried in an oven at 100° C. for 24 hours and then calcined in a box furnace at 500° C. for 4 hours under a constant air flow of 150 mL/min to prepare a Ru/ZrO₂ catalyst on which 5 wt % of Ru was supported. Thereafter, the prepared catalyst was reduced at 300° C. for an additional 2 hours under a 5% H₂/Ar flow of 150 mL/min to activate the catalyst.

### Preparation Example 12: Preparation of 5 wt % Au/ZrO2 Catalyst

Zr(OH)₄ powder was calcined at 600° C. to obtain monoclinic ZrO₂, followed by dispersing 4.75 g of the obtained ZrO₂ in 40 mL of deionized water to prepare a ZrO₂ slurry. Subsequently, 0.5 g was dissolved in 30 mL of deionized water to obtain a gold precursor solution. The obtained gold precursor solution was slowly added to the ZrO₂ slurry under constant stirring to obtain a mixture. The obtained mixture was stirred at room temperature for 6 hours, followed by evaporation of water to recover a solid. The recovered solid was dried in an oven at 100° C. for 24 hours and then calcined in a box furnace at 500° C. for 4 hours under a constant air flow of 150 mL/min to prepare a Au/ZrO₂ catalyst on which 5 wt % of Au was supported. Thereafter, the calcined catalyst was reduced at 300° C. for an additional 2 hours under a 5% H₂/Ar flow of 150 mL/min to activate the catalyst.

### Preparation Example 13: Preparation of 5 wt % Pd/C Catalyst

A Pd/C catalyst (Commercial 205680-50G) on which 5 wt % of Pd was supported, purchased from Sigma-Aldrich, was used.

### Preparation Example 14: Preparation of 5 wt % Rh/C Catalyst

A Rh/C catalyst (Commercial 206164-5G) on which 5 wt % of Rh was supported, purchased from Sigma-Aldrich, was used.

### Experimental Example 1: Analysis of Catalyst Properties

To analyze catalyst properties as a function of the Pt content, the specific surface area, pore size, and pore volume of the catalysts prepared in Preparation Examples 1 and 3 to 5 were analyzed using Brunauer-Emmett-Teller (BET) analysis.

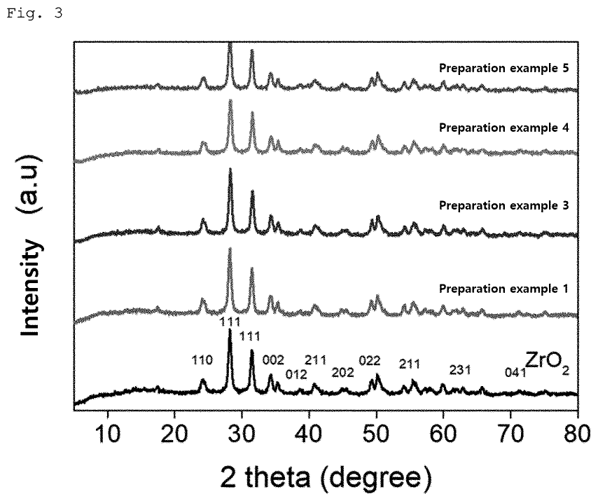

The supported platinum content and size were analyzed using inductively coupled plasma (ICP) analysis and transmission electron microscopy (TEM), respectively. The dispersion of the supported platinum was analyzed by CO chemisorption. The respective results thereof are shown in Table 1. Additionally, XRD patterns were measured to determine the compositions of these catalysts. The results thereof are shown in FIG. 3. In this case, the ZrO₂ of Reference Example 1 in Table 1 below refers to ZrO₂ obtained by calcining Zr(OH)₄ powder at 600° C.

TABLE 1

| | | Surface | Pore | Pore | | TEM | Metal |
|---|---|---|---|---|---|---|---|
| | | Area | Size | Volume | | Particle Size | Dispersion |
| Classification | Catalyst | (m²/g) | (Å) | (cm³/g) | ICP % | (nm) | (%) |
| Reference | ZrO₂ | 32.90 | 341 | 0.28 | — | — | — |
| Example 1 | | | | | | | |
| Preparation | 1 wt % | 33.74 | 360 | 0.30 | 1 | >1 | 72 |
| Example 1 | Pt/ZrO₂ | | | | | | |
| Preparation | 3 wt % | 34.89 | 320 | 0.27 | 2.90 | 1 to | 47 |
| Example 3 | Pt/ZrO₂ | | | | | 2 | |
| Preparation | 5 wt % | 35.65 | 295 | 0.26 | 4.91 | 2 to | 37 |
| Example 4 | Pt/ZrO₂ | | | | | 2.5 | |
| Preparation | 9 wt % | 37.05 | 273 | 0.25 | 8.80 | 2.5 to | 26 |
| Example 5 | Pt/ZrO₂ | | | | | 3 | |

As shown in FIG. 3 and Table 1, in terms of the specific surface area of the catalysts, as well as the platinum content and size, the higher the platinum content, the larger the specific surface area, but the smaller the pore size and pore volume. It was also confirmed that the greater the amount of supported platinum, the lower the platinum dispersion.

### Example: Simultaneous Production of Acid and Alcohol from Sugar

### Example 1

Into a reactor, 0.4 g of the catalyst prepared in Preparation Example 4 (5 wt % Pt/ZrO₂), 2.18 g (12 mmol) of glucose, 0.75 g (12 mmol) of KOH, and 6 mL of distilled water were added, followed by nitrogen gas purging into the reactor to completely replace the air thereinside. Thereafter, the reactor was stirred at 1,400 rpm to carry out a reaction at 25° C. for 6 hours. The reaction product obtained after completion of the reaction was analyzed by liquid chromatography.

### Example 2

Gluconic acid and sorbitol were simultaneously produced from glucose in the same manner as in Example 1, except that the catalyst prepared in Preparation Example 6 (5 wt % Pt/CeO₂) was added to carry out the reaction.

### Example 3

Into a reactor, 0.4 g of the catalyst prepared in Preparation Example 1 (1 wt % Pt/ZrO2), 1.8 g (12 mmol) of xylose, 0.75 g (12 mmol) of KOH, and 6 mL of distilled water were added, followed by nitrogen gas purging into the reactor to completely replace the air thereinside. Thereafter, the reactor was stirred at 1,400 rpm to carry out a reaction at 25° C. for 6 hours. The reaction product obtained after completion of the reaction was analyzed by liquid chromatography.

### Example 4

Xylonic acid and xylitol were simultaneously produced from xylose in the same manner as in Example 3, except that the catalyst prepared in Preparation Example 2 (2 wt % Pt/ZrO₂) was added to carry out the reaction.

### Example 5

Xylonic acid and xylitol were simultaneously produced from xylose in the same manner as in Example 3, except that the catalyst prepared in Preparation Example 3 (3 wt % Pt/ZrO₂) was added to carry out the reaction.

### Example 6

Xylonic acid and xylitol were simultaneously produced from xylose in the same manner as in Example 3, except that the catalyst prepared in Preparation Example 4 (5 wt % Pt/ZrO₂) was added to carry out the reaction.

### Example 7

Xylonic acid and xylitol were simultaneously produced from xylose in the same manner as in Example 3, except that the catalyst prepared in Preparation Example 5 (9 wt % Pt/ZrO₂) was added to carry out the reaction.

### Example 8

Xylonic acid and xylitol were simultaneously produced from xylose in the same manner as in Example 3, except that the catalyst prepared in Preparation Example 6 (5 wt % Pt/CeO₂) was added to carry out the reaction.

### Example 9

Xylonic acid and xylitol were simultaneously produced from xylose in the same manner as in Example 3, except that the catalyst prepared in Preparation Example 7 (5 wt % Pt/Al₂O₃) was added to carry out the reaction.

### Example 10

Xylonic acid and xylitol were simultaneously produced from xylose in the same manner as in Example 3, except that the catalyst prepared in Preparation Example 8 (5 wt % Pt/ZnO) was added to carry out the reaction.

### Example 11

Xylonic acid and xylitol were simultaneously produced from xylose in the same manner as in Example 3, except that the catalyst prepared in Preparation Example 9 (5 wt % Pt/TiO₂) was added to carry out the reaction.

### Comparative Example 1

Gluconic acid and sorbitol were simultaneously produced from glucose in the same manner as in Example 1, except that the catalyst prepared in Preparation Example 10 (5 wt % Pt/C) was added to carry out the reaction.

### Comparative Example 2

Xylonic acid and xylitol were simultaneously produced from xylose in the same manner as in Example 3, except that the catalyst prepared in Preparation Example 11 (5 wt % Ru/ZrO₂) was added to carry out the reaction.

### Comparative Example 3

Xylonic acid and xylitol were simultaneously produced from xylose in the same manner as in Example 3, except that the catalyst prepared in Preparation Example 12 (5 wt % Au/ZrO₂) was added to carry out the reaction.

### Comparative Example 4

Xylonic acid and xylitol were simultaneously produced from xylose in the same manner as in Example 3, except that the catalyst prepared in Preparation Example 13 (5 wt % Pd/C) was added to carry out the reaction.

### Comparative Example 5

Xylonic acid and xylitol were simultaneously produced from xylose in the same manner as in Example 3, except that the catalyst prepared in Preparation Example 14 (5 wt % Rh/C) was added to carry out the reaction.

### Comparative Example 6

Xylonic acid and xylitol were simultaneously produced from xylose in the same manner as in Example 3, except that the catalyst prepared in Preparation Example 14 (5 wt % Ru/C) was added to carry out the reaction.

### Experimental Example 2: Evaluation of Catalytic Activity Upon Simultaneous Production of Gluconic Acid and Sorbitol From Glucose

In the simultaneous production of gluconic acid and sorbitol from glucose, the glucose conversion rate and the yields of gluconic acid and sorbitol in Examples 1 and 2 and Comparative Example 1, in which each reaction was carried out under the same conditions, were measured to evaluate reaction activity according to the catalyst type. The results thereof are shown in Table 2.

TABLE 2

| | | | Glucose | Gluconic | |
|---|---|---|---|---|---|
| | | Pt | Conversion | Acid | Sorbitol |
| | | Content | Rate | Yield | Yield |
| Classification | Catalyst | (wt %) | (%) | (%) | (%) |
| Example 1 | Pt/ZrO₂ | 5 | 99 | 77 | 23 |
| Example 2 | Pt/CeO₂ | 5 | 99 | 73 | 27 |
| Comparative | Pt/C | 5 | 99 | 74 | 25 |
| Example 1 | | | | | |

As shown in Table 2, in all cases of Examples 1 and 2 and Comparative Example 1, the glucose conversion rate was 99% or higher, and the yields of gluconic acid and sorbitol were 73% or higher and 23% or higher, respectively.

### Experimental Example 3: Evaluation of Catalytic Activity Upon Simultaneous Production of Xylonic Acid and Xylitol from Xylose

In the simultaneous production of xylonic acid and xylitol from xylose, the xylose conversion rate and the yields of xylonic acid and xylitol in Examples 6 and 8 to 11 and Comparative Examples 1 to 5, in which each reaction was carried out under the same conditions, were measured to evaluate reaction activity according to the catalyst type. The results thereof are shown in Table 3. In this case, the xylose conversion rate was measured by high-performance liquid chromatography (HPLC). The results thereof are shown in Table 3.

TABLE 3

| | | | Xylose | Xylonic | |
|---|---|---|---|---|---|
| | | Pt | Conversion | Acid | Xylitol |
| | | Content | Rate | Yield | Yield |
| Classification | Catalyst | (wt %) | (%) | (%) | (%) |
| Example 6 | Pt/ZrO₂ | 5 | 100 | 50 | 50 |
| Example 8 | Pt/CeO₂ | 5 | 100 | 50 | 50 |
| Example 9 | Pt/Al₂O₃ | 5 | 100 | 50 | 50 |
| Example 10 | Pt/ZnO | 5 | 100 | 50 | 50 |
| Example 11 | Pt/TiO₂ | 5 | 100 | 50 | 50 |
| Comparative | Pt/C | 5 | 100 | 50 | 50 |
| Example 1 | | | | | |
| Comparative | Ru/ZrO₂ | 5 | 0 | 0 | 0 |
| Example 2 | | | | | |
| Comparative | Au/ZrO₂ | 5 | 0 | 0 | 0 |
| Example 3 | | | | | |
| Comparative | Pd/C | 5 | 50 | 25 | 25 |
| Example 4 | | | | | |
| Comparative | Rh/C | 5 | 53 | 27 | 26 |
| Example 5 | | | | | |
| Comparative | Ru/C | 5 | 0 | 0 | 0 |
| Example 6 | | | | | |

As shown in Table 3, Examples 6 to 11 and Comparative Example 1 exhibited identical selectivity toward xylonic acid and xylitol. In particular, it was shown that the hydrogen utilization efficiency reached the theoretical maximum of 100%.

### Experimental Example 4: Evaluation of Catalytic Activity As Function of Pt Content

In the simultaneous production of xylonic acid and xylitol from xylose, the xylose conversion rate, the yields of xylonic acid and xylitol, and the selectivity toward xylonic acid and xylitol in Examples 3 to 7 were measured to evaluate reaction activity as a function of the catalyst content. The results thereof are shown in Table 4. In this case, the xylose conversion rate was measured by HPLC, and the yields of xylonic acid and sorbitol were calculated at a ratio of 50:49, while the selectivity toward xylonic acid and sorbitol was calculated at a ratio of 50:49.

TABLE 4

| | | | Xylose | Xylonic | Xylonic | | |
|---|---|---|---|---|---|---|---|
| | | Pt | Conversion | Acid | Acid | Xylitol | Xylitol |
| | | Content | Rate | Yield | Selectivity | Yield | Selectivity |
| Classification | Catalyst | (wt %) | (%) | (%) | (%) | (%) | (%) |
| Example 3 | Pt/ZrO₂ | 1 | 90 | 42.5 | 48 | 42 | 47 |
| Example 4 | Pt/ZrO₂ | 2 | 97 | 48 | 49 | 47 | 48 |
| Example 5 | Pt/ZrO₂ | 3 | 98 | 49 | 49 | 48 | 49 |
| Example 6 | Pt/ZrO₂ | 5 | 100 | 50 | 50 | 50 | 50 |
| Example 7 | Pt/ZrO₂ | 9 | 89 | 41 | 47 | 41 | 48 |

As shown in Table 4, as the Pt content supported on the catalyst increased from 1 to 5 wt % with respect to the total weight of the catalyst, the xylose conversion rate, the yields of xylonic acid and xylitol, and the selectivity toward xylonic acid and xylitol increased. On the other hand, it was shown that when the Pt content was 9 wt %, the xylose conversion rate, the yields of xylonic acid and xylitol, and the selectivity toward xylonic acid and xylitol were slightly reduced.

### Experimental Example 5: Evaluation of Catalyst Stability Upon Reuse

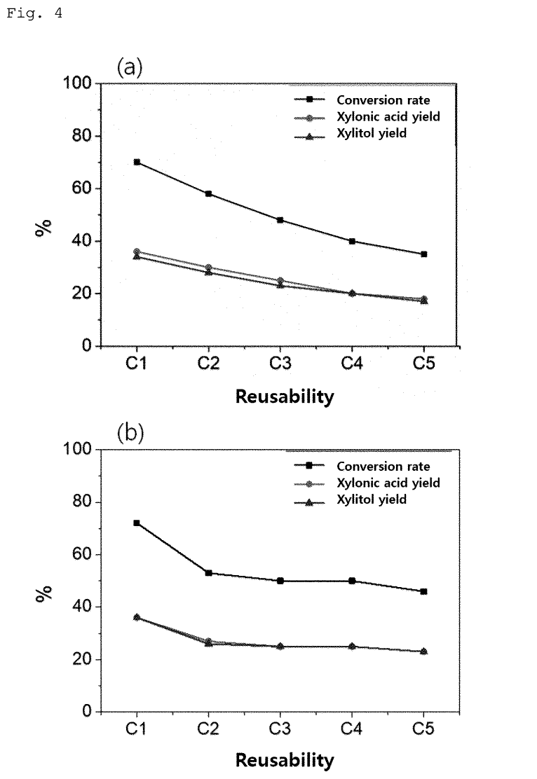

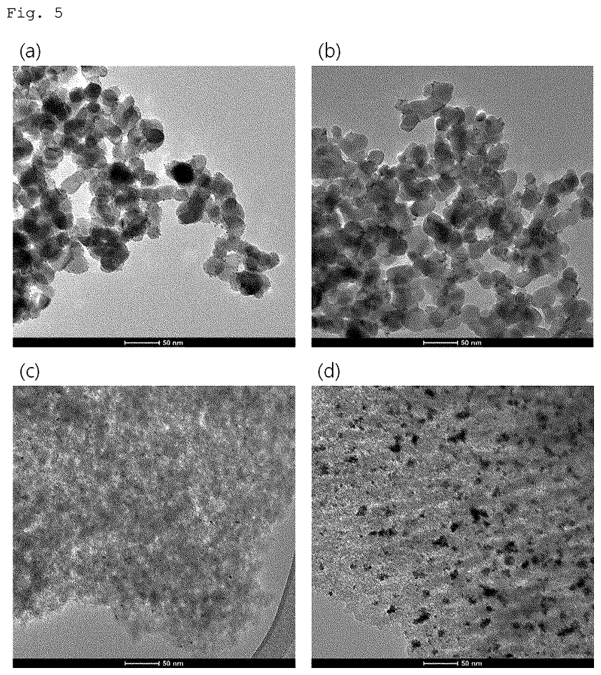

In order to evaluate catalyst stability upon reuse, the catalysts prepared in Preparation Examples 4 and 10 were used to carry out the reaction in the same manner as in Example 6, except that the reaction time was changed from 6 hours to 1 hour. The experiment was repeatedly performed five times to carry out the same reaction again by directly reusing each catalyst used in the reaction. After completion of each reaction, the resulting product was recovered to measure the xylose conversion rate and the yields of xylonic acid and xylitol. The results thereof are shown in FIG. 4. Additionally, SEM images of the catalysts of Preparation Examples 4 and 10 were taken before and after one reaction cycle. The results thereof are shown in FIG. 5.

In FIG. 4, (a) shows a graph illustrating measurements of the xylose conversion rate and the yields of xylonic acid and xylitol for the catalyst of Preparation Example 4, and (b) shows a graph illustrating measurements of the xylose conversion rate and the yields of xylonic acid and xylitol for the catalyst of Preparation Example 10. Additionally, in FIG. 5, (a) shows an SEM image of the catalyst of Preparation Example 4 before the reaction, (b) shows an SEM image of the catalyst of Preparation Example 4 after the reaction, (c) shows an SEM image of the catalyst of Preparation Example 10 before the reaction, and (b) shows an SEM image of the catalyst of Preparation Example 10 after the reaction.

As shown in FIG. 4, in the case of the catalyst prepared in Preparation Example 4, the xylose conversion rate and the yields of xylonic acid and xylitol remained stable even after repeatedly used five times. On the other hand, in the case of the catalyst prepared in Preparation Example 10, the activity continuously decreased, resulting in reduced xylose conversion rate and yields of xylonic acid and xylitol.

Furthermore, even in the SEM measurement shown in FIG. 5, it was confirmed that, in the case of the catalyst of Preparation Example 4, the Pt particle size was maintained after performing the reaction. On the other hand, in the case of the catalyst of Preparation Example 10, Pt particle agglomeration was observed.

Accordingly, it was confirmed that, for the catalyst according to the present disclosure, the reaction activity did not decrease upon reuse, thus demonstrating long-term stability even under basic conditions.

Although the present disclosure has been described above with reference to the accompanying drawings, these embodiments are disclosed only for illustrative purposes. Those skilled in the art to which the present disclosure pertains will appreciate that various modifications and other equivalent embodiments may be made based on the present disclosure. Therefore, the technical scope of protection of the present disclosure should be defined by the appended claims.

1. A catalyst for a simultaneous conversion reaction of a sugar to an acid and an alcohol to simultaneously produce the acid and the alcohol from the sugar in the presence of a base,
wherein the catalyst has a form in which platinum serving as a catalytically active metal is supported on a metal oxide containing one or more metals selected from the group consisting of zirconium (Zr), aluminum (Al), cerium (Ce), zinc (Zn), titanium (Ti), hafnium (Hf), nickel (Ni), cobalt (Co), iron (Fe), manganese (Mn), aluminum (Al), gallium (Ga), and indium (In), the metal oxide serving as a catalyst support.

2. The catalyst of claim 1, wherein the base is present in an amount of 1 mol or more with respect to 1 mol of the sugar.

3. The catalyst of claim 1, wherein the sugar in the reaction has a concentration of 1 mol or more.

4. The catalyst of claim 1, wherein the platinum is contained in an amount in a range of 0.1 to 20 wt % with respect to the total weight of the catalyst.

5. The catalyst of claim 1, wherein the base is a hydroxide of an alkali metal and/or a hydroxide of an alkaline earth metal.

6. The catalyst of claim 1, wherein the sugar is one or more selected from the group consisting of erythrose, ribose, arabinose, xylose, lyxose, allose, altrose, glucose, mannose, galactose, talose, hexose, maltose, lactose, fructose, lactulose, isomaltulose, rhamnose, sucrose, starch sugars, starch hydrolysates, cellulose hydrolysates, and hemicellulose hydrolysates.

7. A method of simultaneously producing an acid and an alcohol from a sugar by subjecting the sugar to a reaction in the presence of a base and a catalyst,
wherein the catalyst wherein the catalyst has a form in which platinum serving as a catalytically active metal is supported on a metal oxide containing one or more metals selected from the group consisting of zirconium (Zr), aluminum (Al), cerium (Ce), zinc (Zn), titanium (Ti), hafnium (Hf), nickel (Ni), cobalt (Co), iron (Fe), manganese (Mn), aluminum (Al), gallium (Ga), and phosphorus (In), the metal oxide serving as a catalyst support.

8. The method of claim 7, wherein the base is present in an amount of 1 mol or more with respect to 1 mol of the sugar.

9. The method of claim 7, wherein the sugar in the reaction has a concentration of 1 mol or more.

10. The method of claim 7, wherein the platinum is contained in an amount in a range of 0.1 to 20 wt % with respect to the total weight of the catalyst.

11. The method of claim 7, wherein the base is a hydroxide of an alkali metal and/or a hydroxide of an alkaline earth metal.

12. The method of claim 7, wherein the reaction is performed at a temperature in a range of 0° C. to 200° C.
